(19) United States
(12) Patent Application Publication
(10) Pub. No.: US 2018/0037865 A1
(43) Pub. Date: Feb. 8, 2018

(54) METHODS AND COMPOSITIONS RELATING TO TREATMENT OF NERVOUS SYSTEM INJURIES

(71) Applicant: Wayne State University, Detroit, MI (US)

(72) Inventors: Jean Peduzzi-Nelson, Northville, MI (US); Harini Sundararaghavan, Grosse Point Farms, MI (US); Assadollah Mazhari, Bloomfield Hills, MI (US)

(21) Appl. No.: 15/670,606

(22) Filed: Aug. 7, 2017

Related U.S. Application Data
(60) Provisional application No. 62/371,526, filed on Aug. 5, 2016.

(51) Int. Cl. C12N 5/0797; A61K 38/39; A61K 47/10; A61K 47/36

(52) U.S. Cl. CPC C12N 5/0623; A61K 47/36; A61K 38/39; A61K 47/10; C12N 5/0619

(57) ABSTRACT

Compositions are provided according to aspects of the present invention which include: a plurality of longitudinally extending fibers, each of the fibers having a longitudinal axis, wherein the longitudinal axis of each of a majority of the fibers is generally aligned; a plurality of stem cells capable of differentiation into a central or peripheral nervous system cell, a majority of the plurality of stem cells in contact with one or more of the plurality of longitudinally extending fibers; a biocompatible hydrogel, wherein the longitudinally extending fibers and stem cells are disposed in the matrix of a biocompatible hydrogel; a therapeutic amount of a neurotrophic factor disposed in the biocompatible hydrogel, wherein the neurotrophic factor is distributed as a gradient; a plurality of olfactory ensheathing cells disposed in the biocompatible hydrogel; and a therapeutic amount of a scar inhibitor disposed in the biocompatible hydrogel.

### REFERENCE TO RELATED APPLICATION

This application claims priority from U.S. Provisional Patent Application Ser. No. 62/371,526, filed Aug. 5, 2016, the entire content of which is incorporated herein by reference.

### FIELD OF THE INVENTION

Methods and compositions described herein are generally related to treatment of nervous system injuries. In specific aspects, methods and compositions are described according to the present invention for use in treatment of chronic severe spinal cord injury in a human.

### BACKGROUND OF THE INVENTION

Nervous system injuries can be among the most devastating, often resulting in significant long-term or permanent functional defects.

There is a continuing need for methods and compositions to treat nervous system injuries.

### SUMMARY OF THE INVENTION

Compositions are provided for treating a nervous system injury in a subject in need thereof according to aspects of the present invention which include: a plurality of longitudinally extending fibers, each of the fibers having a longitudinal axis, a proximal end and a distal end, wherein the longitudinal axis of each of a majority of the fibers is generally aligned with the longitudinal axis of each of the other fibers of the majority; a plurality of stem cells capable of differentiation into a central or peripheral nervous system cell, a majority of the plurality of stem cells in contact with one or more of the plurality of longitudinally extending fibers; a biocompatible hydrogel, wherein the longitudinally extending fibers and stem cells are disposed in the matrix of a biocompatible hydrogel, forming a therapeutic hydrogel structure having a proximal end and a distal end, wherein the proximal end of the majority of the fibers is disposed in the biocompatible hydrogel at the proximal end of the therapeutic hydrogel structure and the distal end of the majority of the fibers is disposed in the biocompatible hydrogel at the distal end of the therapeutic hydrogel structure; a therapeutic amount of a sensory neurotrophic factor disposed in the therapeutic hydrogel structure, wherein the therapeutic amount of the sensory neurotrophic factor is distributed as a gradient, wherein a higher concentration of the neurotrophic factor is present at the proximal end of the therapeutic hydrogel structure, and a lower concentration of the neurotrophic factor is present at the distal end of the therapeutic hydrogel structure and/or a therapeutic amount of a motor neurotrophic factor disposed in the therapeutic hydrogel structure, wherein a lower concentration of the motor neurotrophic factor is present at the proximal end of the therapeutic hydrogel structure, and a higher concentration of the neurotrophic factor is present at the distal end of the therapeutic hydrogel structure; a plurality of olfactory ensheathing cells disposed in the biocompatible hydrogel; and a therapeutic amount of a scar inhibitor disposed in the therapeutic hydrogel structure.

Compositions are provided according to aspects of the present invention wherein the stem cells are olfactory neural stem cells.

Compositions are provided according to aspects of the present invention wherein the neurotrophic factor is selected from the group consisting of: brain-derived neurotrophic factor (BDNF), glial cell-derived neurotrophic factor and a combination thereof.

Compositions are provided according to aspects of the present invention wherein the neurotrophic factor is present in a pharmaceutically acceptable controlled release carrier.

Compositions are provided according to aspects of the present invention wherein the neurotrophic factor is present in pharmaceutically acceptable controlled release microspheres.

Compositions are provided according to aspects of the present invention wherein the stem cells and olfactory ensheathing cells are human stem cells and human olfactory ensheathing cells.

Compositions are provided according to aspects of the present invention wherein the stem cells and olfactory ensheathing cells are human stem cells and human olfactory ensheathing cells obtained from the subject.

Compositions are provided according to aspects of the present invention which include human endothelial cells disposed in the therapeutic hydrogel structure. The endothelial cells form blood vessel structures within the therapeutic hydrogel structure and which are capable of forming connections with endogenous blood vessels once the therapeutic hydrogel structure is implanted in a subject.

Compositions are provided according to aspects of the present invention wherein the biocompatible hydrogel comprises: hyaluronic acid, collagen, fibrin, PEG, chitosan, methylcellulose, silk, small intestinal submucosa or a combination of any two or more thereof.

Optionally, compositions according to aspects of the present invention include a therapeutic hydrogel structure characterized by pores sized to allow passage of materials such as axons, capillaries and other blood vessels. According to aspects of the present invention, the therapeutic hydrogel structure includes a porous hydrogel having pores with diameters in the range of about 4-400 micrometers.

Optionally, one or more additional components included in a therapeutic hydrogel structure is selected from: an RGD sequence (arginine, glycine, aspartate), A blocker of inhibitors of axonal growth such as NOGO, MAG (myelin associated glycoprotein), PTEN (Phosphatase and Tensin Homolog), Rho, Rho-ROCK (Rho kinases), and repulsive guidance molecule (RGM).

Compositions are provided according to aspects of the present invention wherein the fibers comprise or consist of hyaluronic acid.

Compositions are provided according to aspects of the present invention wherein the fibers comprise or consist of polyethylene.

Compositions are provided according to aspects of the present invention wherein the fibers comprise or consist of poly(ethylene oxide)

Compositions are provided according to aspects of the present invention wherein the fibers comprise hyaluronic acid and either polyethylene or poly(ethylene oxide).

Compositions are provided according to aspects of the present invention wherein the fibers comprise cross-linked hyaluronic acid.

Compositions are provided according to aspects of the present invention wherein the fibers comprise hyaluronic acid and poly(ethylene oxide) in a ratio in the range of 80:20-20:80, 75:25-25:75, 70:30-30:70, or 60:40-40:60.

Compositions are provided according to aspects of the present invention wherein the fibers comprise hyaluronic acid and polyethylene in a ratio in the range of 80:20-20:80, 75:25-25:75, 70:30-30:70, or 60:40-40:60.

Compositions are provided according to aspects of the present invention wherein the fibers have a diameter in the range of 1 nm-200 μm or 1 μm-1 mm. Compositions are provided according to aspects of the present invention wherein the fibers have a diameter in the range of 1 μm-200 μm

Compositions are provided according to aspects of the present invention wherein the neurotrophic factor is present in a pharmaceutically acceptable controlled release carrier and the carrier is in contact with the fibers.

Compositions are provided according to aspects of the present invention wherein the scar inhibitor is chondroitinase ABC.

Methods of treating a nervous system injury in a subject in need thereof are provided according to aspects of the present invention which include administering a therapeutic amount of a composition as described herein to a subject at a site of a nervous system injury.

Methods of treating a nervous system are provided according to aspects of the present invention wherein the nervous system injury is a central or peripheral nervous system injury.

Methods of treating a nervous system are provided according to aspects of the present invention wherein the nervous system injury is a spinal cord injury.

Methods of treating a nervous system are provided according to aspects of the present invention wherein the nervous system injury is a chronic severe spinal cord injury.

Methods of treating a nervous system are provided according to aspects of the present invention wherein the subject is human.

### BRIEF DESCRIPTION OF THE DRAWINGS

FIG. 1 is a schematic diagram of an implantable composition according to aspects of the present invention;

FIG. 2A is a scanning electron micrograph image showing non-aligned fibers; scale bar is 10 microns;

FIG. 2B is a scanning electron micrograph image showing aligned electrospun fibers; scale bar is 10 microns;

FIG. 2C is a fluorescence micrograph image showing stem cells growing on the non-aligned fibers in FIG. 2A; scale bar is 100 microns;

FIG. 2D is a fluorescence micrograph image showing stem cells growing on the aligned fibers in FIG. 2B; scale bar is 100 microns;

FIG. 3 is an image showing a hyaluronic hydrogel layered with electrospun aligned hyaluronic fiber scaffolds;

FIG. 4A is a scanning electron micrograph image showing BDNF-releasing and GDNF-releasing poly(lactic-co-glycolic acid) (PLGA) microspheres electrospun to form a gradient of the microspheres on an electrospun hyaluronic acid fiber scaffold;

FIG. 4B is an environmental scanning electron micrograph image showing BDNF-releasing and GDNF-releasing PLGA microspheres electrospun to form a gradient of the microspheres on an electrospun hyaluronic acid fiber scaffold;

FIG. 5 is a graph showing the Basso, Beattie and Bresnahan (BBB) score at various times after surgical implant of an artificial spinal cord (ASC) implant according to aspects of the present invention, wherein a BBB score of 0 indicates complete paralysis and a BBB score of 21 indicates normal locomotion, the * indicates the rats treated by implantation of a segment of ASC is significantly different than segment removal condition (p<0.05). +indicates ASC is significantly different than empty hydrogel condition (p<0.05);

FIG. 6 is a graph showing results of the Plantar Test (Hargreaves' Method) which enables assessment of a peripherally mediated response to thermal stimulation, the * indicates the rats treated by implantation of a segment of ASC is significantly different than segment removal condition (p<0.05). +indicates ASC is significantly different than empty hydrogel condition (p<0.05);

FIG. 7 is a graph showing results of beam walking tests performed, the * indicates the rats treated by implantation of a segment of ASC is significantly different than segment removal condition (p<0.05). +indicates ASC is significantly different than empty hydrogel condition (p<0.05);

FIG. 8 is a graph showing results of an inclined plane test, the * indicates the rats treated by implantation of a segment of ASC is significantly different than segment removal condition (p<0.05). +indicates ASC is significantly different than empty hydrogel condition (p<0.05);

FIG. 9 is a graph showing results of a ladder rung test in which the ASC treated animal group was significantly different than both other conditions.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Kursad Turksen (Ed.), Embryonic stem cells: methods and protocols in Methods Mol Biol. 2002; 185, Humana Press; Current Protocols in Stem Cell Biology, ISBN: 9780470151808; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

Compositions for treating a nervous system injury in a subject in need thereof are provided according to aspects of the present invention which include: 1) an aligned fiber scaffold; 2) a plurality of stem cells capable of differentiation into central or peripheral nervous system cells in contact with the fibers; 3) a therapeutic amount of a sensory and/or motor neurotrophic factor distributed in a gradient along a non-aligned or an aligned fiber scaffold; 4) a plurality of olfactory ensheathing cells; 5) a therapeutic amount of a scar inhibitor; and 6) a biocompatible hydrogel encapsulating all of 1-5.

Compositions for treating a nervous system injury in a subject in need thereof are provided according to aspects of the present invention which include a therapeutic hydrogel structure including a matrix of a biocompatible hydrogel, the matrix coextensive with the therapeutic hydrogel structures and having a proximal and distal end. A pathway directing and encouraging growth of axons from the proximal end of the matrix of biocompatible hydrogel to the distal end is included therein and a pathway directing and encouraging growth of axons from the distal end of the matrix of biocompatible hydrogel to the proximal end is also included. According to aspects of the present invention, the pathway directing and encouraging growth of axons from the proximal end of the matrix of biocompatible hydrogel to the distal end includes a first plurality of aligned or non-aligned fibers and a gradient of a motor neurotrophic factor disposed in contact with the first plurality of aligned or non-aligned fibers. According to aspects of the present invention, the pathway directing and encouraging growth of axons from the distal end of the matrix of biocompatible hydrogel to the proximal end includes a second plurality of aligned or non-aligned fibers and a gradient of a sensory neurotrophic factor disposed in contact with the second plurality of aligned or non-aligned fibers.

According to aspects of the present invention, a third pathway is included which provides a pathway directing and encouraging growth of neurites from stem cells, such as olfactory neural stem cells.

Compositions for treating a nervous system injury in a subject in need thereof are provided according to aspects of the present invention which include: 1) a plurality of longitudinally extending fibers, each of the fibers having a longitudinal axis, a proximal end and a distal end, wherein the longitudinal axis of each of a majority of the fibers is generally aligned with the longitudinal axis of each of the other fibers of the majority; 2) a plurality of stem cells capable of differentiation into a central or peripheral nervous system cell, a majority of the plurality of stem cells in contact with one or more of the plurality of longitudinally extending fibers; 3) a biocompatible hydrogel, wherein the longitudinally extending fibers and stem cells are disposed in the matrix of a biocompatible hydrogel, forming a therapeutic hydrogel structure having a proximal end and a distal end, wherein the proximal end of the majority of the fibers is disposed in the biocompatible hydrogel at the proximal end of the therapeutic hydrogel structure and the distal end of the majority of the fibers is disposed in the biocompatible hydrogel at the distal end of the therapeutic hydrogel structure; 4) a therapeutic amount of a neurotrophic factor disposed in the therapeutic hydrogel structure, wherein the therapeutic amount of the neurotrophic factor is distributed as a gradient, wherein a higher concentration of the neurotrophic factor is present at the proximal end of the therapeutic hydrogel structure, and a lower concentration of the neurotrophic factor is present at the distal end of the therapeutic hydrogel structure or wherein a lower concentration of the neurotrophic factor is present at the proximal end of the therapeutic hydrogel structure, and a higher concentration of the neurotrophic factor is present at the distal end of the therapeutic hydrogel structure; 5) a plurality of olfactory ensheathing cells disposed in the biocompatible hydrogel; and 6) a therapeutic amount of a scar inhibitor disposed in the therapeutic hydrogel structure.

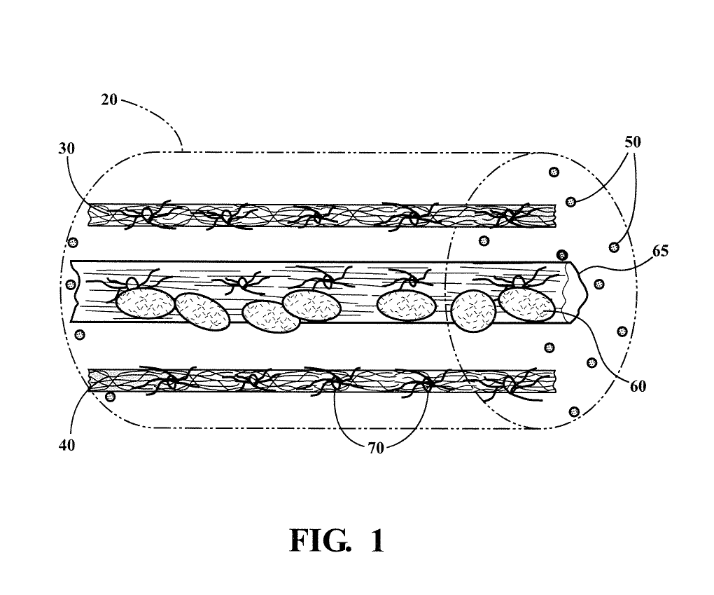

FIG. 1 is a schematic diagram of an implantable composition according to aspects of the present invention. As illustrated, the implantable composition is an “artificial spinal cord” including: a plurality of longitudinally extending fibers 65, each of the fibers having a longitudinal axis, a proximal end and a distal end, wherein the longitudinal axis of each of a majority of the fibers is generally aligned with the longitudinal axis of each of the other fibers of the majority; a plurality of stem cells 60 capable of differentiation into a central or peripheral nervous system cell, a majority of the plurality of stem cells in contact with one or more of the plurality of longitudinally extending fibers; a biocompatible hydrogel 20, wherein the longitudinally extending fibers and associated stem cells are disposed in the matrix of a biocompatible hydrogel, forming a therapeutic hydrogel structure having a proximal end and a distal end, wherein the proximal end of the majority of the fibers is disposed in the biocompatible hydrogel at the proximal end of the therapeutic hydrogel structure and the distal end of the majority of the fibers is disposed in the biocompatible hydrogel at the distal end of the therapeutic hydrogel structure; a therapeutic amount of a first neurotrophic factor disposed in the therapeutic hydrogel structure in association with a plurality of non-aligned or aligned fibers 30, wherein the therapeutic amount of the first neurotrophic factor is distributed as a gradient, wherein a higher concentration of the first neurotrophic factor is present at the proximal end of the therapeutic hydrogel structure, and a lower concentration of the first neurotrophic factor is present at the distal end of the therapeutic hydrogel structure; a therapeutic amount of a second neurotrophic factor disposed in the therapeutic hydrogel structure in association with a plurality of non-aligned or aligned fibers 40, wherein the therapeutic amount of the second neurotrophic factor is distributed as a gradient, wherein a higher concentration of the second neurotrophic factor is present at the distal end of the therapeutic hydrogel structure and a lower concentration of the first neurotrophic factor is present at the proximal end of the therapeutic hydrogel structure; a plurality of olfactory ensheathing cells 70 disposed in the biocompatible hydrogel; and 6) a therapeutic amount of a scar inhibitor 50 disposed in the therapeutic hydrogel structure. According to aspects of the invention, the first neurotrophic factor is GDNF and the second neurotrophic factor is BDNF.

Methods of treating a nervous system injury in a subject in need thereof are provided according to aspects of the present invention which include administering a therapeutic amount of a composition of the present invention to a subject at a site of a nervous system injury. The composition is positioned at the site of the injury so that the proximal end of the therapeutic hydrogel structure is closest to the brain (rostral or superior) the distal end of the therapeutic hydrogel structure that is closest to the conus medullaris (caudal or inferior) axons thereby forming a bridge or scaffold for the neurites to grow and form synapses. The nervous system injury to be treated can be a central or peripheral nervous system injury. According to aspects of the present invention, the nervous system injury is a spinal cord injury. According to aspects of the present invention, the nervous system injury is a chronic severe spinal cord injury.

While compositions and methods are described herein with emphasis on humans, the subject to be treated can be any animal, preferably a mammal or bird, including, human, non-human primates, rats, mice, guinea pigs, cats, dogs, horses, cattle, pigs, goats, sheep, rabbits and birds.

“Treating” or “treatment” to achieve a beneficial effect in a subject having an injury of the nervous system describes the administration of compositions of the present invention in an amount and for a time sufficient to achieve the benefit of the compositions, such as an alleviation, in whole or in part, of symptoms associated with the injury, or a slowing, inhibition or halting of further progression or worsening of those symptoms, or an actual improvement of the injured nervous system. For example, within the context of treating severe spinal cord injury, successful treatment may include one or more clinical benefits, an alleviation of symptoms, such as stabilization or improvement in movement ability, coordination and/or sensation, or slowing or halting the progression of neurodegeneration, as measured by a stabilization, reduction or halt in progressive loss of movement ability, coordination and/or sensation.

An inventive composition may be administered acutely or chronically. For example, a composition as described herein may be administered once or several times at varying intervals depending on the particular characteristics of the subject and the injury to be treated. One of skill in the art could determine an appropriate treatment schedule in view of these and other considerations typical in medical practice.

Fibers

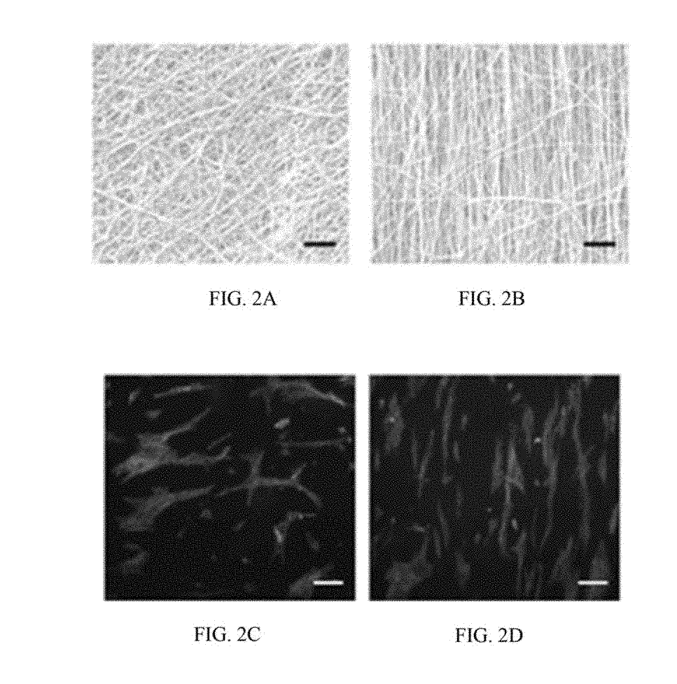

Fibers included in compositions for treating a nervous system injury in a subject in need thereof are aligned to direct the growth of processes of neurons, for example, so the axons can ascend or descend in a straight line as the axons do in a normal spinal cord. FIGS. 2A-D show that cells grown on aligned fibers extend their processes in a straight line compared to cells grown on non-aligned fibers.

The fibers are elongated and have a longitudinal axis and the longitudinal axes of the majority of fibers in the composition are aligned with each other. The term “majority” refers to greater than 50%, such as 60%, 70%, 80%, 90% or greater.

Fibers can be aligned, for example, by electrospinning methods of making the fibers.

Fibers included in compositions for treating a nervous system injury in a subject in need thereof include one or more of: hyaluronic acid collagen, gelatin, PCL, PLA, PLGA, fibrin, small intestinal submucosa, and silk, according to aspects of the present invention.

According to further aspects, the fibers include both hyaluronic acid and polyethylene and optionally, cross-linked hyaluronic acid and polyethylene.

According to aspects in which the fibers include hyaluronic acid and polyethylene, the hyaluronic acid and polyethylene are present in a ratio in the range of 80:20-20:80, such as 70:30-30:70 or 60:40-40:60. According to aspects of the present invention, the fibers include or consist of hyaluronic acid.

According to aspects of the present invention, the fibers include or consist of polyethylene.

According to aspects of the present invention, the fibers include or consist of poly(ethylene oxide).

According to further aspects, the fibers include both hyaluronic acid and poly(ethylene oxide) and optionally, cross-linked hyaluronic acid and poly(ethylene oxide).

According to aspects in which the fibers include hyaluronic acid and poly(ethylene oxide), the hyaluronic acid and poly(ethylene oxide) are present in a ratio in the range of 80:20-20:80, such as 70:30-30:70 or 60:40-40:60.

The fibers can have a diameter in the range of 1 nm-200 μm, optionally in the range of 1 nm-1 μm or 1 μm-200 μm but can have a larger or smaller diameter.

The length of included fibers depends on the application and can be in the range of about 100 μm-1 cm, but may be longer or shorter.

The fibers can be synthesized and aligned by various methods. A preferred method of synthesizing and aligning the fibers is by electrospinning which allows control over mechanics, adhesion, porosity, and fiber alignment, aiding in organizing and aligning neurites.

According to aspects of the present invention, one or more additional populations of fibers are present which are not aligned or wherein less than the majority of fibers are aligned. In particular aspects, fibers which support a gradient of a neurotrophic factor, such as a neurotrophic factor incorporated in controlled-release particles, may be aligned or non-aligned.

Stem Cells

Included stem cells are those capable of differentiating into cells found in the central or peripheral nervous system, preferably neurons and/or glial cells. According to aspects of the present invention, the stem cells are neural stem cells. Neural stem cells are well known in the art as multipotent cells characterized by their ability to differentiate into neurons, astrocytes and oligodendrocytes in response to exposure to appropriate stimuli.

According to aspects of the present invention, stem cells included in the compositions are olfactory neural stem cells. The normal fate of these cells is to become neural cells, which reduces the risk in transplanting into the nervous system. A virtually pure population of olfactory mucosa neural stem cells can be obtained by selective culture of cells from olfactory mucosa tissue that lines the uppermost part of the nose. Two populations of stem cells in the olfactory mucosa exist that can develop into neurons, placode-derived basal epithelial cells and neural crest-derived mesenchymal lamina propria cells. If desired, one or both of these types of olfactory neural stem cells can be purified for use in compositions according to aspects of the present invention. In a non-limiting example, magnetically activated cell sorting can be used to differentiate and isolate placode-derived basal epithelial cells and/or neural crest-derived mesenchymal lamina propria cells. According to preferred aspects, where the subject is human, the stem cells used are human olfactory neural stem cells. According to further preferred aspects, where the subject is human, the stem cells are olfactory neural stem cells obtained from the subject.

Biocompatible Hydrogel

A biocompatible hydrogel is included as a matrix which surrounds and contains components including fibers, stem cells, olfactory ensheathing cells, growth factors, and scar inhibiting agent.

The term “biocompatible” as used herein refers to a composition that is substantially non-toxic in vivo and which does not cause deleterious effects to the materials contained in the hydrogel or in a subject implanted with the composition.

The term “hydrogel” as used herein refers to a three-dimensional network of molecules which are covalently bound, in chemical hydrogels, or non-covalently associated, in physical hydrogels, and where water is the liquid component of the hydrogel. Mixtures of chemical hydrogels and physical hydrogels are encompassed by the term “hydrogel” as used herein.

Cross-linking of polymers can be used to modify properties of the hydrogel. For example, hyaluronic acid (HA) can be readily modified on its hydroxyl group with methacrylation and photo-crosslinked to form a hydrogel. Varying degrees of methacrylation can be used to control the mechanical and degradation characteristics of the hydrogels.

Optionally, compositions according to aspects of the present invention include a therapeutic hydrogel characterized by pores seized to allow passage of materials such as axons, capillaries and other blood vessels. Porosity of a therapeutic hydrogel can be controlled by various methods such as, but not limited to, varying the degree of cross-linking, photo patterning, laser patterning, adding matrix metalloproteinases (MMPS), using a polymer degradable once implanted to form the therapeutic hydrogel structure.

According to aspects of the present invention, a biocompatible, photocrosslinkable hydrogel is included. The biocompatible hydrogel can include, but is not limited to, hyaluronic acid, collagen, fibrin, PEG, chitosan, methylcellulose, silk, small intestine submucosa, or a combination of any two or more thereof.

Optionally, mixtures of polymers are used in a biocompatible hydrogel as a matrix in a therapeutic hydrogel structure wherein the polymers have different rates of degradation when implanted in a subject. For example, at least one included polymer has a shorter rate of degradation so that its degradation results in pores in the hydrogel. For example, collagen and fibrin degrade relatively quickly (for instance compared to hyaluronic acid). In a further example, polymers including ester bonds are readily degraded in vivo.

In a non-limiting example, to create the therapeutic hydrogel structure, a mixture of one or more polymers and cross-linker is placed in a mold as a semi-solid then the other components of the composition are added and the mixture is solidified.

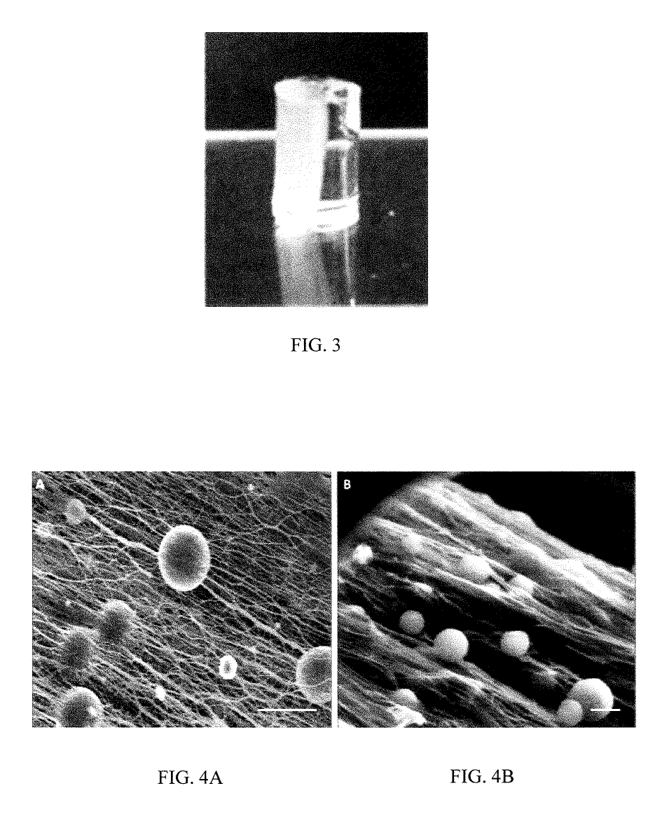

In a particular example, the composition is an “artificial spinal cord” and the 1) aligned fiber scaffold; 2) plurality of stem cells capable of differentiation into central or peripheral nervous system cells in contact with the fibers; 3) therapeutic amount of a neurotrophic factor distributed in a gradient along the aligned fiber scaffold; 4) plurality of olfactory ensheathing cells; and 5) therapeutic amount of a scar inhibitor; are held together in a hydrogel that is approximately the same size as the spinal cord to be treated (see FIG. 3).

Hyaluronic acid (HA) hydrogels are included according to aspects of the present invention. HA is a naturally found non-adhesive, biocompatible polysaccharide that is made up of alternating D-glucuronic acid and N-acetyl-D-glucosamine that is found in most connective tissues. HA is enzymatically degradable and important in many cellular functions including attachment, proliferation, and migration. HA hydrogels support cell survival and can degrade by natural enzymes

Therapeutic Amount of a Neurotrophic Factor

Compositions are provided according to aspects of the present invention wherein a neurite guidance factor is presented in a gradient to direct and encourage growth of neurites towards the proximal or distal end of the therapeutic hydrogel structure.

According to aspects of the present invention, a “motor neurotrophic factor” is disposed in a gradient to direct and encourage growth of neuronal processes toward the distal end of the therapeutic hydrogel structure. The gradient of the “motor neurotrophic factor” has a higher concentration at the distal end of the therapeutic hydrogel structure and a lower concentration at the proximal end of the therapeutic hydrogel structure. The term “motor neurotrophic factor” is used herein to refer to a substance which encourages directional growth of axons, particularly an axon of a motor neuron. Examples of motor neurotrophic factors include, but are not limited to, BDNF, GDNF, neurturin, nodal, and persephin, and mixtures of any two or more thereof.

According to aspects of the present invention, a “sensory neurotrophic factor” is disposed in a gradient to direct and encourage growth of neuronal processes toward the proximal end of the therapeutic hydrogel structure. The gradient of the “sensory neurotrophic factor” has a higher concentration at the proximal end of the therapeutic hydrogel structure and a lower concentration at the distal end of the therapeutic hydrogel structure. The term “sensory neurotrophic factor” is used herein to refer to a substance which encourages directional growth of axons, particularly an axon of a sensory neuron. Examples of sensory neurotrophic factors include, but are not limited to, BDNF, GDNF, acetylcholine, nerve growth factor, and mixtures of any two or more thereof.

A neurotrophic factor included in compositions according to aspects of the present invention is selected from the group consisting of: brain-derived neurotrophic factor (BDNF), glial cell-derived neurotrophic factor (GDNF) and a combination thereof. BDNF is a member of the neurotrophin family that encourages the growth of axons in the brain. Gradients of BDNF direct the axonal growth of both sensory and motor neurons, primarily motor neurons. GDNF mainly enhances sensory axonal regeneration, having an influence on motor axons but a greater influence on sensory neurons.

Neurotrophic factors can be isolated from sources in which they naturally occur, can be produced using molecular recombinant techniques or can be chemically synthesized, using well-known methods.

A therapeutically effective amount of a sensory or motor neurotrophic factor included in a composition according to the present invention has a beneficial effect on nervous system cells and tissues at or near the site of injury such as stimulating neuronal process outgrowth and inhibiting neuronal death. The amount will vary depending on the severity of the condition to be treated, the species of the subject, the age and sex of the subject and the general physical characteristics of the subject to be treated. One of skill in the art could determine a therapeutically effective amount in view of these and other considerations typical in medical practice. In general it is contemplated that a therapeutically effective amount would be in the range of about 0.001 mg/kg-100 mg/kg body weight, optionally in the range of about 0.01-10 mg/kg, and further optionally in the range of about 0.1-5 mg/kg.

Optionally, an included sensory or motor neurotrophic factor is associated with a pharmaceutically acceptable controlled release carrier. Controlled release compositions for inclusion of a sensory or motor neurotrophic factor include, but are not limited to, polymer particles such as polymer microparticles or nanoparticles of any of various shapes such as microspheres or nanospheres. Controlled release compositions are well-known and pharmaceutically acceptable carriers and formulation of sustained, intermittent or otherwise regulated release formulations in pharmaceutical compositions are known in the art, illustratively including, but not limited to, as described in Remington: The Science and Practice of Pharmacy, 21^{st} Ed., Lippincott, Williams & Wilkins, Philadelphia, Pa., 2006; Allen, L. V. et al., Ansel's Pharmaceutical Dosage Forms and Drug Delivery Systems, 8^{th} Ed., Lippincott, Williams & Wilkins, Philadelphia, Pa., 2005; M. Mishra, Handbook of Encapsulation and Controlled Release, CRC Press, Taylor & Francis Group, 2016; and Siepmann et al. (eds), Fundamentals and Applications of Controlled Release Drug Delivery, Springer, 2012.

In a non-limiting example, poly(lactic-co-glycolic acid) (PLGA) microspheres are loaded with BDNF for inclusion in an inventive composition.

In a non-limiting example, PLGA microspheres are loaded with GDNF for inclusion in an inventive composition.

According to aspects of the present invention, a therapeutic amount of a factor is distributed as a gradient in the biocompatible hydrogel. Compositions are provided according to aspects of the present invention wherein a factor is presented in a gradient to specifically direct axons of sensory or motor neurons.

According to aspects of the present invention, a higher concentration of GDNF is present at the proximal end of the therapeutic hydrogel structure, and a lower concentration of the GDNF is present at the distal end of the therapeutic hydrogel structure.

According to aspects of the present invention, a lower concentration of BDNF is present at the proximal end of the therapeutic hydrogel structure, and a higher concentration of the BDNF is present at the distal end of the therapeutic hydrogel structure.

According to aspects of the present invention, the neurotrophic factor is formulated as a pharmaceutically acceptable controlled release composition. According to preferred aspects, the neurotrophic factor is formulated for controlled release from a particulate controlled release composition which is distributed in a gradient along the proximal to distal axis of the therapeutic hydrogel structure. According to further aspects of the present invention, the neurotrophic factor is formulated for controlled release from a particulate controlled release composition which is distributed in a gradient along the proximal to distal axis of the therapeutic hydrogel structure in contact with the fibers included in the therapeutic hydrogel structure.

Distribution of a growth factor along a gradient is achieved, for example, by inclusion of a growth factor associated with a pharmaceutically acceptable solid support carrier, such as a particulate carrier, in a solution of the fiber polymer and subjecting the mixture to electrospinning, resulting in aligned or non-aligned fibers distributed along a length of a support and a gradient of the growth factor associated with a pharmaceutically acceptable solid support carrier distributed along the support with the aligned or non-aligned fibers.

Alternative methods for growth factor delivery include blend electrospinning, emulsion electrospinning and core-shell electrospinning. Other neurotrophic factors can be used to direct the growth of sensory and motor axons.

Olfactory Ensheathing Cells

Olfactory ensheathing cells (OECs) myelinate both the axonal processes of differentiated transplanted stem cells and also the ascending and descending host axons. OECs are efficient at migrating to the sites of damage and form compact myelin with restoration of impulse conduction in the demyelinated spinal cord.

According to preferred aspects, where the subject is human, the olfactory ensheathing cells used are human olfactory neural cells. According to further preferred aspects, where the subject is human, the olfactory ensheathing cells are olfactory ensheathing cells obtained from the subject.

Endothelial Cells

Compositions are provided according to aspects of the present invention which include human endothelial cells disposed in the therapeutic hydrogel structure. The endothelial cells form blood vessel structures within the therapeutic hydrogel structure and which are capable of forming connections with endogenous blood vessels once the therapeutic hydrogel structure is implanted in a subject.

According to further preferred aspects, where the subject is human, the endothelial cells are obtained from the subject.

Therapeutic Amount of a Scar Inhibitor

One or more scar inhibiting agents are included to prevent and/or inhibit scar formation and/or reduce a scar structure, including, but not limited to, chondroitinase ABC, retinoic receptor agonists, deferoxamine mesylate, and histamine.

Chondroitinase ABC is an enzyme catalyzes the eliminative degradation of polysaccharides containing 1,4-β-D-hexosaminyl and 1,3-β-D-glucuronosyl or 1,3-α-L-iduronosyl linkages to disaccharides containing 4-deoxy-b-D-gluc-4-enuronosyl groups, such as degrading chondroitin sulphate proteoplycans in scar tissue.

Chondroitinase ABC can be isolated from an organism which produces it naturally, such as P. vulgaris, or it can be produced using molecular recombinant techniques or by chemical synthetic methodology.

A therapeutically effective amount of chondroitinase ABC included in a composition according to the present invention has a beneficial effect of degrading at least a portion of scar tissue that may be present at the site of an injury. The amount will vary depending on the severity of the condition to be treated, the species of the subject, the age and sex of the subject and the general physical characteristics of the subject to be treated. One of skill in the art could determine a therapeutically effective amount in view of these and other considerations typical in medical practice. In general it is contemplated that a therapeutically effective amount would be in the range of about 0.001 mg/kg-100 mg/kg body weight, optionally in the range of about 0.01-10 mg/kg, and further optionally in the range of about 0.1-5 mg/kg.

According to aspects of the present invention, the scar inhibitor is dissolved in the hydrogel (e.g., at 0.5 U/500 μl) before adding other components.

Embodiments of inventive compositions and methods are illustrated in the following examples. These examples are provided for illustrative purposes and are not considered limitations on the scope of inventive compositions and methods.

### EXAMPLES

Olfactory Stem Cells

The olfactory tissues can be obtained in a minimally invasive method from inside the nose as described in Escada et al., 2009, Eur Arch Otorhinolaryngol. 266(11):1675-8015.

Inbred Lewis rats are overdosed with urethane (0.42 ml/100 g body wt. of 10.8 g in 19.2 ml.) until dead. The head is detached and skin and subcutaneous tissue is removed. The anterior skull and snout is cut at the parasagittal plane (1.5 mm lateral to midline) with a 22 blade on number 4 scalpel handle. The olfactory mucosa is separated from the olfactory mucosa using an 11 blade. Sterile fine Dumont forceps are used to transfer the tissue using into a petri dish containing one drop of sterile Hank's balanced salt solution on an ice pack. The tissue is minced into small pieces using 2 number 11 blades. The cell suspension is placed into a 1.5 ml sterile tube then spun at 60 rcf for 5 minutes. The supernatant is removed then 2 ml of Dispase and 2.3 microliters of DNase II is added and incubated for 30 minutes at 37 degrees Celsius. The tube is then spun at 60 rcf for 5 minutes and pellet resuspended in 1.5 ml of Accutase. After 30 minutes of incubation at 37 degrees Celsius, the pellet is resuspended in DMEM/F12 media with 10% fetal calf serum. After 2 days in a 5% CO2 humidified incubator, the culture media with cells is spun at 60 rcf for 10 minutes and pellet resuspended at 20,000-80,000 cells/ml in DMEM/F12 with 2% B27, basic fibroblast growth factor (20 ng/ml), epidermal growth factor (20 ng/ml) with 1% Pen/Strep or 1% antibiotic/antimycotic (AbAm). Media is usually changed every other day or when cells reach a density of 200,000/ml. When sufficient cells were available, free-floating cells are spun at 60 rcf for 5 minutes and the cell pellet is resuspended in 0.5 ml DMEM/F12. Cells are plated onto the electrospun fibers that are glued to 12 mm coverslips in 100 mm tissue culture dishes. Stem cells are allowed to attach for at least 3 days in culture before including in the ASC. For preparing human olfactory stem, a similar procedure is followed.

Positive magnetic cell sorting is now used so a fixed 4:1 ratio of c-Kit and THY-1 cells can be used. For magnetic labeling and separation protocol, BD™ IMag Buffer is diluted 1:10 with sterile distilled water. The cell suspension is centrifuged at 60 rcf for 5 minutes and the supernatant is completely removed.

The pellet is resuspended cells in buffer up to 10̂7 nucleated cells per 200 ml buffer. The biotinylated antibody is added at the appropriate concentration (20 ul for CD90), mixed well, and incubated for 15 minutes in the dark in the refrigerator. The labeled cells are washed by adding 5 mL of 1×BD IMag™ buffer and centrifuge at 60 rcf for 5 minutes and supernatant removed. For the positive selections the BD IMag™ Streptavidin Particles Plus—DM are vortexed thoroughly and 20 μl of particles is added for every 1×10̂7 total cells. After mixing thoroughly, the cells and particles are refrigerated for 30 minutes at 6° C.-12° C. then the labeling volume is brought up to 2 to 8×10̂7 cells/ml with 1×BD IMag™ buffer. The tube is placed onto the Cell Separation Magnet and incubated for 6 to 8 minutes.

With the tube on the Cell Separation Magnet, the supernatant (considered the negative fraction) is aspirated. The tube is removed from the Cell Separation Magnet, and 1×BD IMag™ buffer is added to the same volume. The cells are gently resuspended by pipetting up and down, and the tube is returned to the Cell Separation Magnet for another 2 to 4 minutes the supernatant is again removed. This separation is repeated again. After the final wash step, the tube is removed from the Cell Separation Magnet and the positive fraction is resuspended in culture medium for further expansion.

Olfactory Ensheathing Cells (OECs)

A pure population of olfactory ensheathing cells can be obtained based on selective attachment in defined media from olfactory bulb or the olfactory mucosa tissue that lines the uppermost part of the nose as described in Sasaki et al., J Neurosci. 2004 Sep. 29; 24(39):8485-93. The cell suspension is prepared as described for neural stem cells. To obtain OECs, cells are plated on poly-l-lysine coated plates in DMEM/10% fetal bovine serum. Non-adherent cells are discarded to obtain a nearly pure population of OECs as described in Sasaki et al., J Neurosci. 2006 Feb. 8; 26(6):1803-12. Magnetic activated cell sorting is optionally used to get a defined population using antibodies specific for the neurotrophin receptor. OECs are allowed to attach to electrospun fibers with gradients of GDNF- and BDNF-releasing microspheres for at least three days prior to inclusion in the ASC. These cells are capable of extensive migration and therefore can migrate into the surrounding hydrogel.

Fibers

Electrospinning fabrication of aligned fibers is described in this example.

In order to fabricate electrospun hyaluronic acid (HA) scaffolds, 2 wt % HA, 3 wt % PEO (poly(ethylene oxide) carrier polymer) and 0.05% Irgacure 2959 (crosslinker) are dissolved in distilled water. Electrospinning out of water, allows inclusion of proteins and growth factors into the scaffold without affecting viability. The polymer solution is electrospun by pumping this solution at 1.2 ml/hr through a 22 kV charged needle towards a grounded rotating mandrel (2 inch diameter) at a distance of 15 cm. When the mandrel is spinning at ≧10 m/s, fibers are aligned. The electrospun fibers are attached to a sterile coverslip using methacrylate and placed in a small sterile petri dish. Then olfactory progenitor cells at 40,000 cells/ml in DMEM/F13 with 2% B27 with 1% Pen/Strep or 1% antibiotic/antimycotic (AbAm) are added and incubated with the aligned fibers.

Additional methods for electrospinning fibers are described in Sundararaghavan et al., Biomacromolecules, 2011, 12(6):2344-50; and Sundararaghavan et al., Biotechnol Bioeng. 2013, 110(4):1249-54.

Growth Factor Gradients

Compositions are provided according to aspects of the present invention wherein a factor is presented in a gradient to specifically direct axons of sensory or motor neurons.

Growth factor gradients are established in compositions according to aspects of the present invention by including gradients of BDNF releasing and GDNF-releasing poly(lactic-co-glycolic acid) (PLGA) microspheres. Microspheres containing neurotrophic factors slowly release the factors over at least a 60 day period using this formulation.

The growth-factor laden PLGA microspheres are incorporated into electrospun HA aligned fiber scaffolds according to aspects of the present invention. PLGA microspheres are fabricated through a double emulsion of 65:35 or 75:25 PLGA in dichloromethane and polyvinyl alcohol (PVA) in water. Prior to electrospinning, microspheres were dispersed in the electrospinning solution (distilled water, MeHA, PEO, 12959) at a concentration of 200 mg microspheres/ml ˜5 ng BDNF/ml or GDNF) and electrospun using the same electrospinning parameters described above. Scaffolds are evaluated using both scanning electron microscopy (SEM) and environmental scanning electron microscopy (ESEM) shown in FIGS. 4A-4B. Brain derived neurotrophic factor (BDNF) and GDNF are included in the inner PVA and water layer of the microspheres. Gradient presentation is achieved by offsetting electrospinning spinnerets on the collection mandrel by 6 cm similar to methods described in Baker et. al., Biomaterials, 2008. 29(15): p. 2348-58. Following gradient formations, scaffolds are rotated and a layer of aligned fibers are spun on top of the gradient scaffolds. The scaffolds are marked to electrospinning, indicating the region of highest concentration of microspheres. Prior to electrospinning, microspheres are dispersed in the electrospinning solution (DI water, MeHA, PEO, 12959) at 200 mg/ml. One solution contains BDNF and one contains GDNF in order to create a gradient of BDNF and GDNF. GDNF is used to guide axons of sensory neurons so highest concentration of GDNF-containing beads is placed rostrally (closer to the brain, i.e. at the proximal end of the fibers and therapeutic structure) in the rats. BDNF is used to guide axons of descending motor command neurons so highest concentration of BDNF-containing beads is placed caudally (closer to the tail, i.e. at the distal end of the fibers and therapeutic structure) in the rats. Scaffolds containing opposing GDNF and BDNF microsphere gradients are used for the top and bottom layers in the scaffold.

Preparation of Hydrogel and Placement or Inclusion of Components in Hyaluronic Acid Hydrogel

Hyaluronic acid (HA) hydrogel is prepared in this example. HA can be readily modified on its hydroxyl group with methacrylation and photo-crosslinked to form a hydrogel (Burdick et al., 2005, Biomacromolecules 6(1):386-91). Varying degrees of methacrylation can be used to control the mechanical and degradation characteristics of the hydrogels (Bencherif et al. 2008, Biomaterials 29(12):1739-49; Burdick et al., 2005, Biomacromolecules 6(1):386-91). In order to control the methacrylation percentage on the backbone, 1 wt. % sodium hyaluronate (molecular weight--40 kDa, ECM Science, Detroit, Mich.) is first dissolved in DI water and reacted with methacrylic anhydride at a 20 fold excess and pH=8 for 24 hours on ice. After the reaction is complete, methacrylated HA is dialyzed in water for three days with at least six solution changes to remove unreacted components and lyophilized. The percent methacrylation (percentage of HA repeat units with a methacrylate group) can be adjusted by altering methacrylic anhydride concentration and evaluated through ¹H NMR (Bruker Advance 360 MHz, Bruker), and this controls the mechanics of the final scaffold. In order to make the final hydrogel, 3% HA is dissolved in DI water with 10% I2959 photocrosslinker. The chondroitinase ABC (0.5 U/500 μl HA) and olfactory ensheathing cells (10,000 cells/ml HA) are added to HA. The HA is placed as a semi-solid in a mold that consists of a plastic 1 cc syringe that has a V-shape cut out of syringe (length wise) on top. The components of the artificial spinal cord (aligned electrospun fibers with attached olfactory neural progenitor cells, 2 aligned electrospun fibers that have gradients of microspheres that slowly release neurotrophic factors with attached OECs) are added, then the V-shape piece of plastic is replaced. Ends of the electrospun fibers with the highest concentration of specific neurotrophic releasing microspheres are marked so orientation can be maintained, and then the HA is photocrosslinked under UV-light for 30 mins to solidify the hydrogel. The coverslips are marked to show the orientation to place the electrospun fibers in the correct direction.

Surgical Procedure

For pre-emptive analgesia, the rats are given Buprenex (0.01-0.05 mg/kg SC) 30 minutes before surgery. Rats (250-300 g) are anesthetized using an isoflurane induction chamber (5% isoflurane with oxygen at 1 liter/minute) until they go down, after which they are maintained under anesthesia with isoflurane (1.5-2% with oxygen at 1 liter/minute using a calibrated vaporizer and a nose cone). Ointment is applied to the eyes to prevent drying. The back of the animal is shaved with clippers then cleaned three times with 7.5% povidone-iodine solution followed by 70% alcohol (repeated 3 times). The bladder is expressed. To express the bladder, the top of the bladder is palpated then gentle pressure is applied until a drop of urine is seen. The pressure at the same level is continued until the bladder is almost empty. The bladder is never completely emptied. The animal is then placed on a water circulating heating pad covered with a sterile towel on the surgery table and draped with sterile towels so that only the surgical region is exposed. Aseptic procedures are followed for the surgery: only autoclaved, sterile instruments, gauze, applicator sticks, etc. are used. For subsequent surgeries on the same day, surgical instruments are soaked in 70% alcohol for 10 minutes, and then rinsed in sterile saline. The surgeons wear masks and sterile gloves. During the surgery, the breathing rate of the animal is monitored almost continuously. The level of isoflurane is increased if there is any response to pain or discomfort, such as increased respiratory rate, response to paw pinch. An incision is made dorsal to the spinal column and thoracic vertebrae exposed from T6 to T13. Laminectomies are performed from T7 or T8 to T10 or T11 using a Leica Neurosurgical Microscope and high power drill. Care is taken to leave the ligaments between the spinous processes intact. The dura is incised just 1 mm lateral to the dorsal vein after placing stitches through the dura lateral to the proposed incision. Visible vessels are cauterized. A segment of spinal cord (5 mm) is removed using microscissors after transecting the cord rostrally and caudally. The cavity is cleared using mild suction. To ensure that the entire spinal cord was removed, the inner surface of the dura was scraped with the edge of a bent 25 gauge needle. The artificial spinal cord (therapeutic hydrogel structure composition according to aspects of the present invention including: 1). Hyaluronic acid hydrogel; 2). Electrospun fibers; 3). Gradients of microspheres (beads) that slowly release 2 types of neurotrophic growth factors cells; 4). Olfactory stem cells; 5). Olfactory ensheathing cells; and 6). Chondroitinase ABC) is cut to the dimensions of the cavity of the spinal cord and implanted in the cavity. The dura are closed with two to four 8-0 or 9-0 sutures. Durafilm (Surgicel) are placed on the surface of the spinal cord. A sterile stainless surgical steel wire (25 gauge is bent in a hairpin shape and cut to 2 cm length and width of 0.24 cm) are bent and then be placed on the surface of the lamina close to the spinous processes. The purpose of the wire is to stabilize the spine so scoliosis does not develop. If the wire is not used, the vertebral column usually bends extensively (dorsally or laterally) at the site of injury and/or treatment. The muscles are sutured with 4-0 Vicryl then the skin is closed using wound clips. The rat is given 5 cc of saline subcutaneously to compensate for any fluid loss.

Post-op (see below for additional post-op care): The rat is kept on oxygen and monitored until fully alert. Rats are placed in individual isolator (filter on top) cages with a water-circulating heating pad under the bottom of half of the cage and given Lactated Ringers twice a day for 1-2 postoperatively. A second dose of Buprenex (0.01-0.05 mg/kg SQ) is administered 6-12 hrs after the pre-emptive dose and the day after surgery. Rimadyl (5 mg/kg SC daily) is administered once daily for 2 days post-operatively. Baytril (10 mg/kg SC) is administered twice daily for 7 days after surgery as a prophylactic antibiotic to prevent urinary bladder infections. Animals have water bottles that have longer sipper tubes and an additional bottle with AIN-76 (Bioserv). Food pellets and treats (kale, corn on the cob, cereal, soft chow, watermelon) are placed in a dish on the floor of the cage. Rats are weighed daily during the first week and then weekly. Wound clips are removed 7-10 days after surgery. Cages are kept partially on a heating pad. Bladders are expressed 2-3 times/day until bladder function returns.

Results

The artificial spinal cord was implanted in the cavity and the dura was closed with very fine suture (9-0). Four groups of rats were used: 1). sham (laminectomy only, no damage to spinal cord) and spinal segment removal groups that received: 2). no graft; 3). hydrogel only; and 4). artificial spinal cord. Rats were blindly tested each week for 15 weeks using the BBB locomotor test that is a 21 point scale with 21 being normal and 0 equal to no movements. The rats were observed in an open area for 4 minutes and scored. The rats were also tested using a modified Hargreaves device that measures how long it takes for the rat to move its paw after warming the hind footpad by shining a light on it. A rat that has decreased feeling in the footpad would take longer to move its foot and a rat with increased sensitivity to pain would move its foot more rapidly. One of the concerns with any spinal cord treatment is increased sensitivity to even mildly painful stimuli.

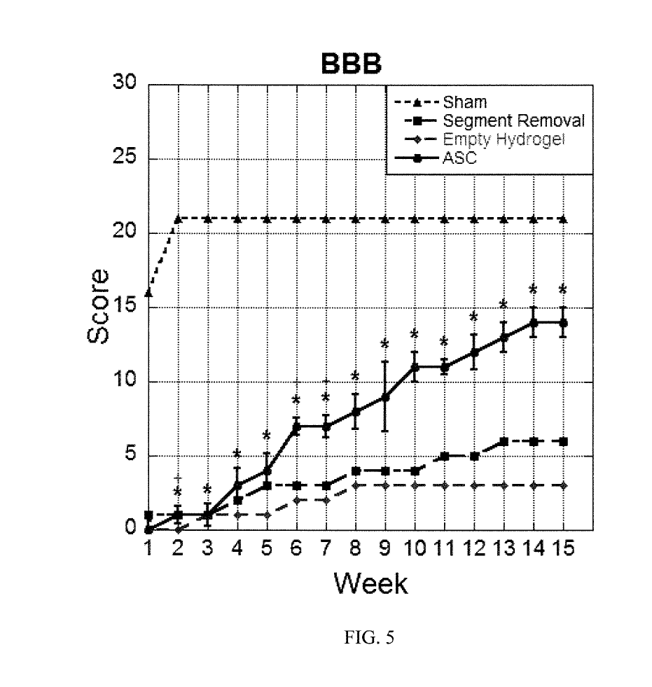

The results of the 3 rats that received the artificial spinal cord showed that the rats went from having a severe injury to a mild injury. In the BBB scale, an important milestone is the gain of hindlimb support that is a score of 9 and gain of forelimb-hindlimb coordination that is a score of 15. The rats that received an artificial spinal cord almost improved to a score of 15 as shown in the graph below (FIG. 5).

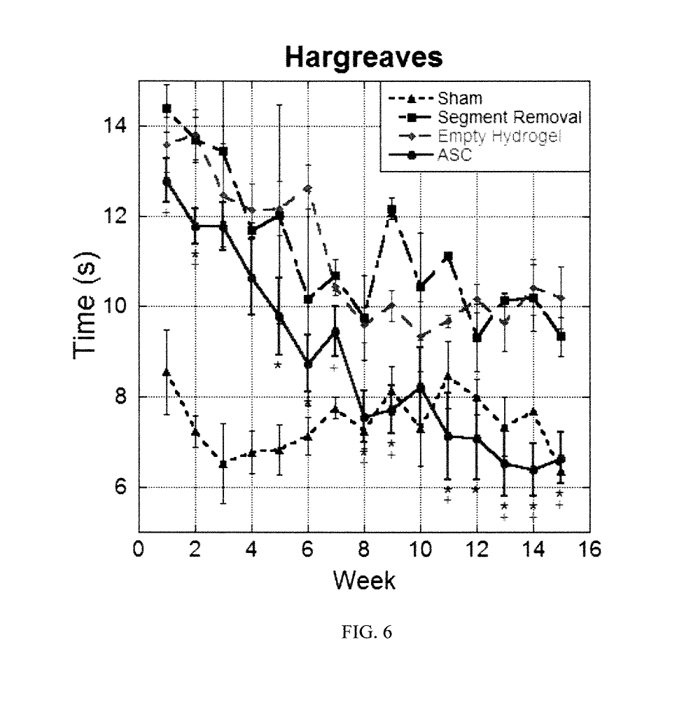

In the Hargreaves test, result shown in FIG. 6, the rats that received the artificial spinal cord reacted to mildly painful stimulus similar to the sham rats that only had some bone removed to reveal the spinal cord. Sham rats had no damage to the spinal cord.

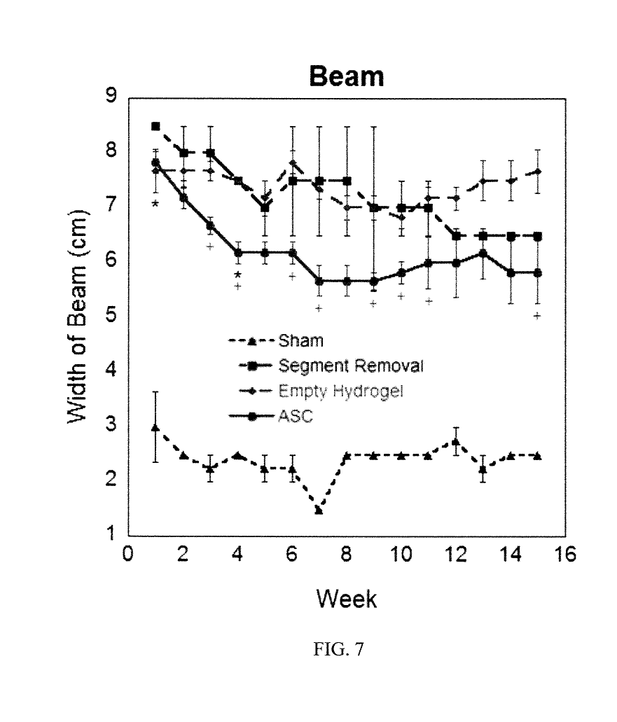

In the Beam test, result shown in FIG. 7, the rats with artificial spinal cords performed significantly better than the other injured groups.

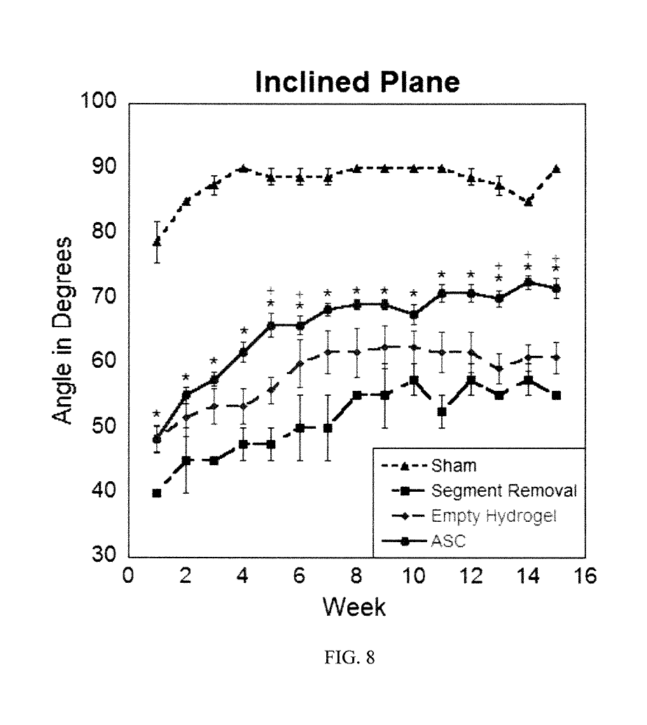

In the Inclined Plane test, result shown in FIG. 8, rats receiving the artificial spinal cord could maintain their balance at a higher angle than the other 2 injured groups at two weeks.

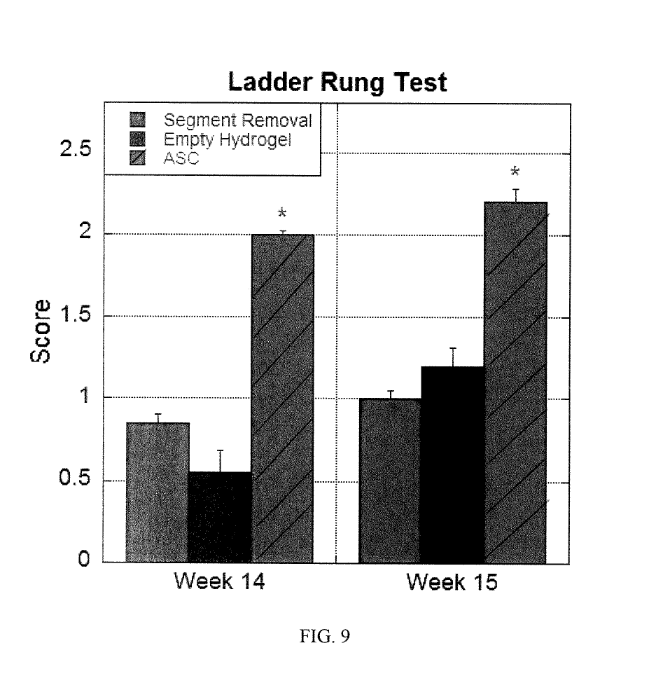

For the ladder test, the ladder had unequal-spaced rungs that indicate use of the corticospinal pathway. This was measured at 14 and 15 weeks following injury. All of the rats were placed on the ladder and the number of footfalls was evaluated. In the ladder rung test, scoring was performed according to Metz & Whishaw (Journal of visualized experiments: JoVE, 2009). A total of 5 steps were evaluated in each of the 2 trials, the mean was given for testing at weeks 14 and 15. On a 0-7 scale, 0 is when the rung is completely missed while 7 is correct placement. The group that received the artificial spinal cord had the highest score on this test (FIG. 9).

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A composition for treating a nervous system injury in a subject in need thereof, comprising:
a plurality of longitudinally extending fibers, each of the fibers having a longitudinal axis, a proximal end and a distal end, wherein the longitudinal axis of each of a majority of the fibers is generally aligned with the longitudinal axis of each of the other fibers of the majority;
a plurality of stem cells capable of differentiation into a central or peripheral nervous system cell, a majority of the plurality of stem cells in contact with one or more of the plurality of longitudinally extending fibers;
a biocompatible hydrogel, wherein the longitudinally extending fibers and stem cells are disposed in the matrix of a biocompatible hydrogel, forming a therapeutic hydrogel structure having a proximal end and a distal end, wherein the proximal end of the majority of the fibers is disposed in the biocompatible hydrogel at the proximal end of the therapeutic hydrogel structure and the distal end of the majority of the fibers is disposed in the biocompatible hydrogel at the distal end of the therapeutic hydrogel structure;
a therapeutic amount of a neurotrophic factor disposed in the therapeutic hydrogel structure, wherein the therapeutic amount of the neurotrophic factor is distributed as a gradient, wherein a higher concentration of the neurotrophic factor is present at the proximal end of the therapeutic hydrogel structure, and a lower concentration of the neurotrophic factor is present at the distal end of the therapeutic hydrogel structure or wherein a lower concentration of the neurotrophic factor is present at the proximal end of the therapeutic hydrogel structure, and a higher concentration of the neurotrophic factor is present at the distal end of the therapeutic hydrogel structure;
a plurality of olfactory ensheathing cells disposed in the biocompatible hydrogel; and
a therapeutic amount of a scar inhibitor disposed in the therapeutic hydrogel structure.

2. The composition of claim 1, wherein the stem cells are olfactory neural stem cells.

3. The composition of claim 1, wherein the neurotrophic factor is selected from the group consisting of: brain-derived neurotrophic factor (BDNF), glial cell-derived neurotrophic factor and a combination thereof.

4. The composition of claim 1, wherein the neurotrophic factor is present in a pharmaceutically acceptable controlled release carrier.

5. The composition of claim 4, wherein the neurotrophic factor is present in pharmaceutically acceptable controlled release microspheres.

6. The composition of claim 1, wherein the stem cells and olfactory ensheathing cells are human stem cells and human olfactory ensheathing cells.

7. The composition of claim 1, wherein the stem cells and olfactory ensheathing cells are human stem cells and human olfactory ensheathing cells obtained from the subject.

8. The composition of claim 1, wherein the biocompatible hydrogel comprises: hyaluronic acid, collagen, fibrin, PEG, chitosan, methylcellulose or a combination of any two or more thereof.

9. The composition of claim 1, wherein the fibers comprise hyaluronic acid.

10. The composition of claim 9, wherein the fibers comprise hyaluronic acid and either polyethylene or poly(ethylene oxide).

11. The composition of claim 10, wherein the fibers comprise cross-linked hyaluronic acid.

12. The composition of claim 10, wherein the fibers comprise hyaluronic acid and poly(ethylene oxide) in a ratio in the range of 80:20-20:80, 70:30-30:70, or 60:40-40:60.

13. The composition of claim 1, wherein the fibers have a diameter in the range of 1 nm-200 μm or 1 μm-200 μm.

14. The composition of claim 1, wherein the neurotrophic factor is present in a pharmaceutically acceptable controlled release carrier and the carrier is in contact with the fibers.

15. The composition of claim 1, wherein the scar inhibitor is chondroitinase ABC.

16. A method of treating a nervous system injury in a subject in need thereof, comprising:
administering a therapeutic amount of a composition according to claim 1 to a subject at a site of a nervous system injury.

17. The method of claim 16, wherein the nervous system injury is a central or peripheral nervous system injury.

18. The method of claim 16, wherein the nervous system injury is a spinal cord injury.

19. The method of claim 18, wherein the nervous system injury is a chronic severe spinal cord injury.

20. The method of claim 16, wherein the subject is human.
